(19) United States
(12) Patent Application Publication
(10) Pub. No.: US 2007/0098135 A1
(43) Pub. Date: May 3, 2007

(54) Method for the reconstruction of a tomographic representation of an object

(76) Inventor: Holger Kunze, Bubenreuth (DE)

Correspondence Address: HARNESS, DICKEY & PIERCE, P.L.C., P.O.BOX 8910, RESTON, VA, 20195, US

(21) Appl. No.: 11/585,920

(22) Filed: Oct. 25, 2006

(30) Foreign Application Priority Data
Oct. 27, 2005 (DE) 10 2005 051 620.3

(51) Int. Cl. H05G 1/60; A61B 6/00; G01N 23/00; G21K 1/12

(52) U.S. Cl. 378/4

(57) ABSTRACT

A method is disclosed for the iterative analytical reconstruction of a tomographic representation of an object from projection data of a moving radiation source through this object onto a detector. In an embodiment of the method, corrections are undertaken iteratively with the aid of back projections of the object to be represented from calculated projection data, wherein the corrections are performed on the projections.

### Priority Statement

The present application hereby claims priority under 35 U.S.C. §119 on German patent application number DE 10 2005 051 620.3 filed Oct. 27, 2005, the entire contents of which is hereby incorporated herein by reference.

### FIELD

The invention generally relates to a method for the iterative analytical reconstruction (ART) of a tomographic representation of an object from projection data of a moving radiation source through this object onto a detector. For example, it may relate to one in which corrections are undertaken iteratively from calculated projection data with the aid of back projections of the object to be displayed in the reconstruction method.

### BACKGROUND

Computed tomography (CT) provides a diagnostic and measuring method for medicine and test engineering with the aid of which internal structures of a patient or test object can be examined without needing in the process to carry out surgical operations on the patient or to damage the test object. In this case, there are recorded from various angles, a number of projections of the object to be examined from which it is possible to calculate a 3D description of the object.

It is generally known to solve this problem by using the so called filtered back projection (Filtered Back Projection FBP), the following documents being referenced by way of example: Buzug: “Einführung in die Computertomographie”, 1st edition 2004, Springer-Verlag, ISBN 3-540-20808-9 and Kak, Slaney: “Principles of Computerized Tomographic Imaging”, 1987, IEEE Press, ISBN 0-87942-198-3. FBP is a high performance computing method in which measured projections are filtered and back projected onto the image. In this method, the image quality depends on the applied filters or convolution cores. These can be specified exactly in analytical terms for simple scanning geometries. Essentially, these are circular paths in the case of which many projections are recorded in uniform angular steps. More complex recording geometries that violate these assumptions lead to problems when attempting to determine the filters analytically. An example of this is tomosynthesis, where in the most general case only a few projections are obtained on a free path from a restricted angular range.

Iterative methods such as the algebraic reconstruction method (ART) have proved their worth for such reconstruction problems. Reference is made in this regard to the following documents: Buzug: “Einführung in die Computertomographie”[“Introduction to computer tomography”], 1st edition 2004, Springer-Verlag, ISBN 3-540-20808-9; Kak, Slaney: “Principles of Computerized Tomographic Imaging”, 1987, IEEE Press, ISBN 0-87942-198-3 and T. Wu, J. Zhang, R. Moore, E. Rafferty, D. Kopans, W. Meleis, D. Kaeli: “Digital Tomosynthesis Mammography Using a Parallel Maximum Likelihood Reconstruction Method”, Medical Imaging 2004: Physics of Medical Imaging, Proceedings of SPIE Vol. 5368 (2004) 1-11.

Iterative methods are based on the principle that the measured projections are compared with the projections calculated from the already reconstructed object, and that the error is subsequently used for the correction of the image of the object. In this process, the image in the nth iteration Xₙ is calculated with the aid of the update equation
Xₙ=Xₙ₋₁+RV(Y−PXₙ₋₁). equ.(1)

There is a suitable initial image X₀, for example a zero image, at the start of the iteration. P in this case represents the system matrix with the aid of which the projections are calculated from the scanned object image using knowledge of the scanning geometry. V is a conditioning matrix with the aid of which the convergence rate can be influenced. In the simplest case, it is a diagonal matrix with identical values, for example the value 1.

Convergence acceleration can be achieved when V corresponds to a convolution of the difference projections with the aid of a ramp filter. A very good reconstruction is possible in this case with 3 iterations.

The computing time required to enable calculation of equ.(1) can be calculated as follows: firstly, there is a need to calculate the projections, this being followed by determining the difference between calculated projection and measured projection, and a back projection of the data lastly being carried out onto the volume. If the calculation of the difference is neglected and the times for calculating the projection and back projection are equated, twice the time for the back projection is required for calculating an iteration.

Because of its iterative nature, the entire computing time is found to last twice the number of iterations multiplied by the time for a filtered back projection.

Since even a simple back projection lasts a relatively long time as a matter of course, the computing time required in the case of iterative back projections constitutes a great impediment to their use.

It is true that the dissertation by Mueller K.: “Fast and accurate three-dimensional reconstruction from Cone-Beam projection data using Algebraic Methods”, Ohio State Univ., 1998 discloses an improved iterative reconstruction method that is based on a solution employing graphics cards, but this method still requires twice the number of iterations multiplied by the time for a filtered back projection, and is therefore still too slow for practical clinical application.

### SUMMARY

In at least one embodiment of the invention, an iterative reconstruction method is described that accomplishes the task of reconstruction in a short computing time.

The inventor has realized, in at least one embodiment, that a method for the iterative calculation of tomographic representations that saves time by comparison with the prior art and in which the multiple projections and back projections are worked through can be carried out when the computational steps of the projection and back projection are performed simultaneously or in parallel with one another for the entire display. This is rendered possible by virtue of the fact that the projections and back projections are no longer carried out in image-wise fashion, but in a pixel-wise or voxel-wise or channel-wise fashion. It is true that the projection and back projection are still calculated serially with reference to a pixel, but these calculations can be split up into a number of processes in a voxel-wise, parallelized fashion such that a rapid acceleration occurs.

The precise mathematical principle is supplied further below in the description of the figures. The computing time can be halved by comparison with the conventional implementation by way of this parallelization. If, furthermore, the error in the comparison between the recorded projections and the calculated forward projections is ramp filtered in the iteration before being used for the correction, it is possible to calculate a filtered back projection in approximately three times the time.

In accordance with the above finding, the inventor proposes, in at least one embodiment, to improve the method known per se for the iterative analytical reconstruction (ART) of a tomographic representation of an object from projection data of a moving radiation source through this object onto a detector, in the case of which corrections are undertaken iteratively in the reconstruction method with the aid back projections of the object to be represented from calculated projection data, this being done by performing the corrections on the projections.

In a preferred design of at least one embodiment of the method, for the iterative process

projections of the object are recorded and at least one representation of the object is back projected,

forward projections are calculated from the at least one tomographic representation of the object,

the recorded projections and the forward projections are compared with one another,

the difference values appearing here between the recorded projections and the calculated forward projections are used as correction values for a corrected projection, and

subsequently the corrected projections are used for renewed calculation of a tomographic representation of the object, forward projections therefrom and the difference values between the recorded projections and the calculated forward projections and the corrected projection is corrected therewith, until the absolute values of the difference values or the number of the iterations reaches a respectively prescribed maximum value.

The correction should preferably be performed exclusively on the projections.

This method according to at least one embodiment of the invention now also renders it possible to perform the back projections and the forward projections in parallel and in a fashion offset by channel or—when an appropriate assignment is performed in advance—to carry out the back projections and the forward projections in parallel and in a voxel-wise or pixel-wise fashion.

It is, moreover, advantageous when difference projections are calculated in the comparison between the recorded projections and the calculated forward projections, and the difference projections are ramp filtered before the correction of the corrected projections. The number of the iteration steps, and thus also the computing time can be substantially reduced thereby.

According to at least one embodiment of the invention, when calculating the back projections of various corrected projections use is made of a number of arithmetic logic units that is smaller than the number of corrected projections performed.

It is also possible that when calculating the back projections of various corrected projections use is made of a number of arithmetic logic units that is the same as the number of corrected projections performed.

Moreover, it is advantageous with reference to an optimized computing time when the calculation of the back projections of consecutive voxels or pixels is carried out on various arithmetic logic units. The sequence of the voxels is generally of subordinate importance. It is customary to use that which is present in the memory. It is to be assumed that it is also possible to find a sequence in the case of which consecutive voxels are as far as possible not mapped on the same projections in the case of spiral paths, as a result of which an acceleration can be attained here once again.

Furthermore, the calculation of the forward projections can be performed by a number of arithmetic logic units that is smaller than the number of forward projections to be calculated, or the calculation of the forward projections can be carried out by the same number of arithmetic logic units as for the forward projections to be calculated.

Given appropriate sorting, it is also possible to carry out the calculation of the forward projections of consecutive voxels or pixels on various arithmetic logic units.

In accordance with the above-described fundamental idea of the method according to at least one embodiment of the invention, the inventor also proposes a tomography unit in the case of which projections are obtained from x-ray imaging, there being present in this process and executed during operation programs that carry out the method steps as claimed in at least one of the preceding method claims. As an alternative, it is also possible to use the tomography unit to obtain projections from magnetic resonance imaging, from ultrasound imaging or from optical imaging without departing from the framework of at least one embodiment of the invention.

### BRIEF DESCRIPTION OF THE DRAWINGS

The invention, in particular also the mathematical principles for the improved reconstruction method, are described below in more detail, using example embodiments, with the aid of the figures, only the features required to understand the invention being illustrated. Use is made for this purpose of the following reference numerals: 101: x ray source at a first position; 101′: x ray source at another position; 102: x ray beam of a first projection; 102′: x ray beam of another projection; 103: detector at a first position; 103′: detector at another position; 104: reconstruction field; 105: evaluation computer; 106: display unit; 107: memories for filters; 108: object/patient; 201: measured projection; (forward projection); 202: back projector; 203: tomographic display; 204: projector (calculation of the projections); 205: calculated projection; 206: subtraction; 207: difference projection; 208: decision unit for truncation of the iteration; 209: back projector for difference projection; 210: difference image; 211: finished image; 301: measured projection; 302: copying operation; 303: corrected projections; 304: back projection; 305: image of the object; 306: projector (calculation of the projections from the object); 307: calculated projections; 308: subtraction carried out between calculated projections and measured projections; 309: difference projections; 310: decision unit for truncation of the iteration; 311: filtering of the difference projections; 312: filtering of the original projections; 313: finished image; 401: distribution computer; 402-404: arithmetic logic units; 405: calculated projection; 501-503, 505-507: projection; 504 and 508: arithmetic logic unit; 506: summation of the results of the back projectors; 601: measured projections; 602: back projector; 603: provisionally reconstructed object; 604: projector; 605: subtraction; 606: summation of measured projections and calculated projections; 607: buffer for measured projections; 608: corrected projections of the first iteration; 609: back projector; 610: provisionally reconstructed object; 611: projector; 612: subtraction; 613: summation of measured projections and calculated projections; 614: corrected projections of the second iteration; 615: back projector; 618: reconstruction result (object); Prg_{x:} programs.

In detail:

FIG. 1: shows a typical CT arrangement with an x ray source;

FIG. 2: shows a flowchart of the known ART method;

FIG. 3: shows a flowchart of the ART method according to an embodiment of the invention;

FIG. 4: shows a flowchart of the ART method according to an embodiment of the invention with parallel processing;

FIG. 5: shows a flowchart of the projection-wise parallelization of the back projection;

FIG. 6: shows a flowchart of the iteration-wise pipeline of the ART method.

### DETAILED DESCRIPTION OF THE EXAMPLE EMBODIMENTS

The terminology used herein is for the purpose of describing particular embodiments only and is not intended to be limiting of the present invention. As used herein, the singular forms “a”, “an” and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing example embodiments illustrated in the drawings, specific terminology is employed for the sake of clarity. However; the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described.

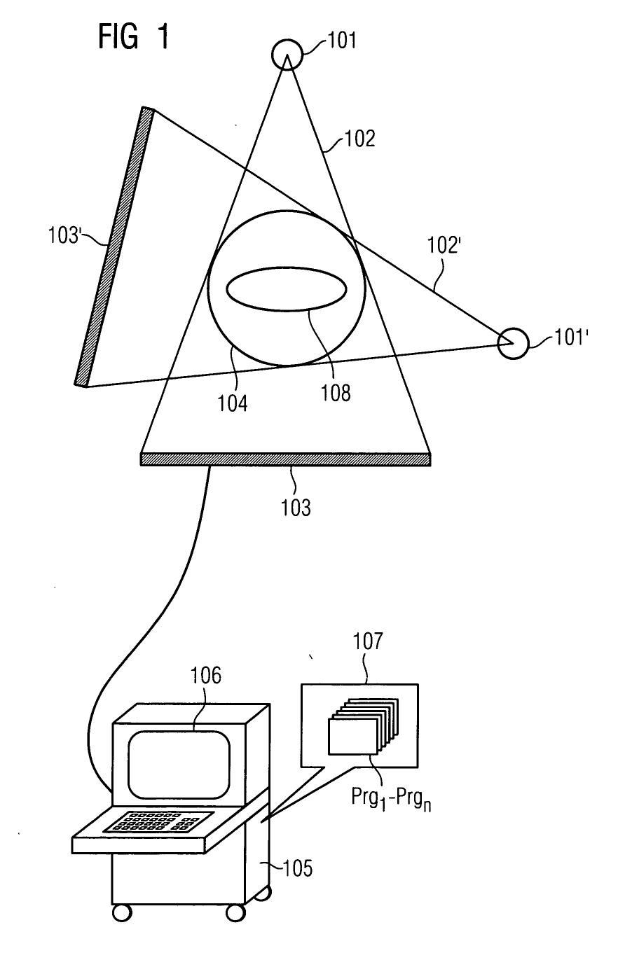

FIG. 1 shows a known typical CT arrangement with an x ray source 101, in a first position, that emits for a first projection an x ray beam 102 that is detected in a detector 103 at this first position after it has penetrated the object, here a patient 108, lying in the reconstruction field 104 and to be examined. The data of the detector pass into an evaluation computer 105 that undertakes the reconstruction, and are subsequently displayed on a display unit 106. The x ray source 101 moves here in an ideal way on a circular path, numerous projections being recorded from different angles. The x ray source 101′ is also illustrated in FIG. 1 in another angular position, the x ray beam 102′ being emitted for another projection that is then detected in the detector 103′ at this other position.

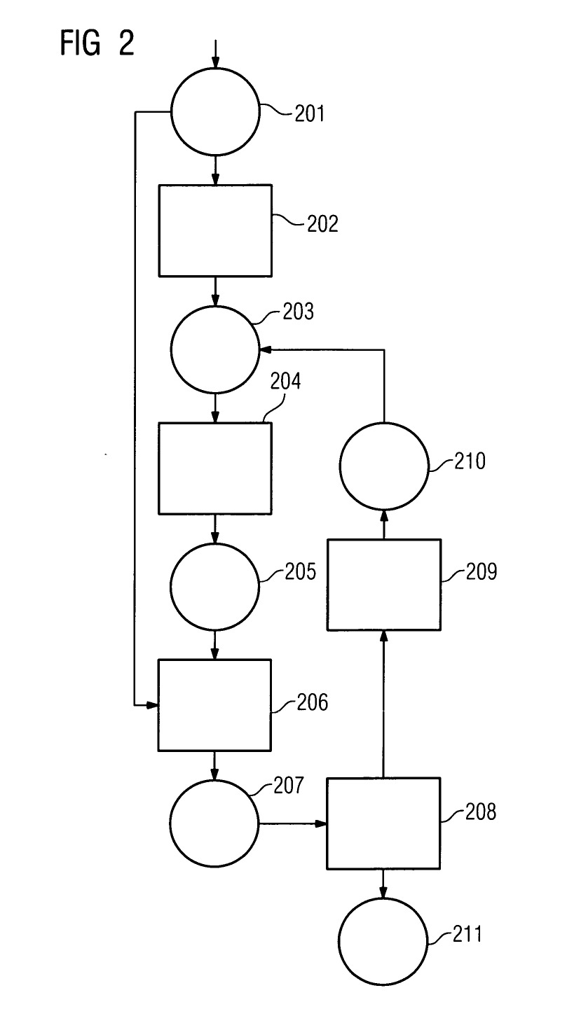

FIG. 2 describes the conventional implementation of an iterative reconstruction; in step 202, the measured projections (forward projections) 201 are back projected onto the object to be reconstructed, in more precise terms the tomographic representation thereof. The image 203 is obtained as a result. Subsequently, after all the back projections have been prepared, forward projections 205 of the object to be reconstructed are calculated in step 204. Subsequently, the difference between the calculated forward projections 205 and the measured projections 201 is calculated in step 206, and the difference projections 207 result.

A decision is made in step 208 as to whether the deviation between the measured projections 201 and the forward projections 205 calculated from the back projected image 203 is sufficiently small, or a decision is made as to whether a sufficiently large number of iteration passes have been performed. If the difference is still too large, or a sufficient number of iterations have not yet been performed, a difference image 210 is prepared in step 209 by back projection from the difference projections 207. This difference image 210 is added to the image 203 for correction. The result is a corrected image 203.

Subsequently, the forward projections from the corrected image 203 are recalculated, and the algorithm passes to the next iteration. The calculation is terminated when the error is sufficiently small, or a specific number of iterations is reached. The reconstructed object, the corrected image 211, is then present in the memory of the computer.

In this implementation, the computing time per iteration is the sum of the computing times for the projection and the back projection. The time required for the remaining computing steps can in general be neglected.

According to an embodiment of the invention, this method is modified and method steps are arranged in a different fashion. The mathematical foundation for this is set forth below:

The description of ART described in equ.(1), which is common in the literature, can be rewritten as follows, Xₙ₋₁ being introduced as a back projection of “corrected data” Yₙ₋₁. The result is:
Xₙ₋₁=RYₙ₋₁, equ.(2)
such that equ. (2) can be rewritten as follows:

$$\begin{matrix}{X}_{n} = R {Y}_{n - 1} + R V ( Y - P R {Y}_{n - 1} ) \\ = R ( {Y}_{n - 1} + V ( Y - P R {Y}_{n - 1} ) ) \\ = R ( ( 1 - V P R ) {Y}_{n - 1} + V Y )\end{matrix} \qquad equ . ( 3 )$$

It follows for Yₙ that

$$\begin{matrix}{Y}_{n} = {Y}_{n - 1} + V ( Y - P R {Y}_{n - 1} ) \\ = {Y}_{n - 1} + V Y - V P R {Y}_{n - 1}\end{matrix} \qquad equ . ( 4 )$$

Yₙ is referred to as corrected projection below.

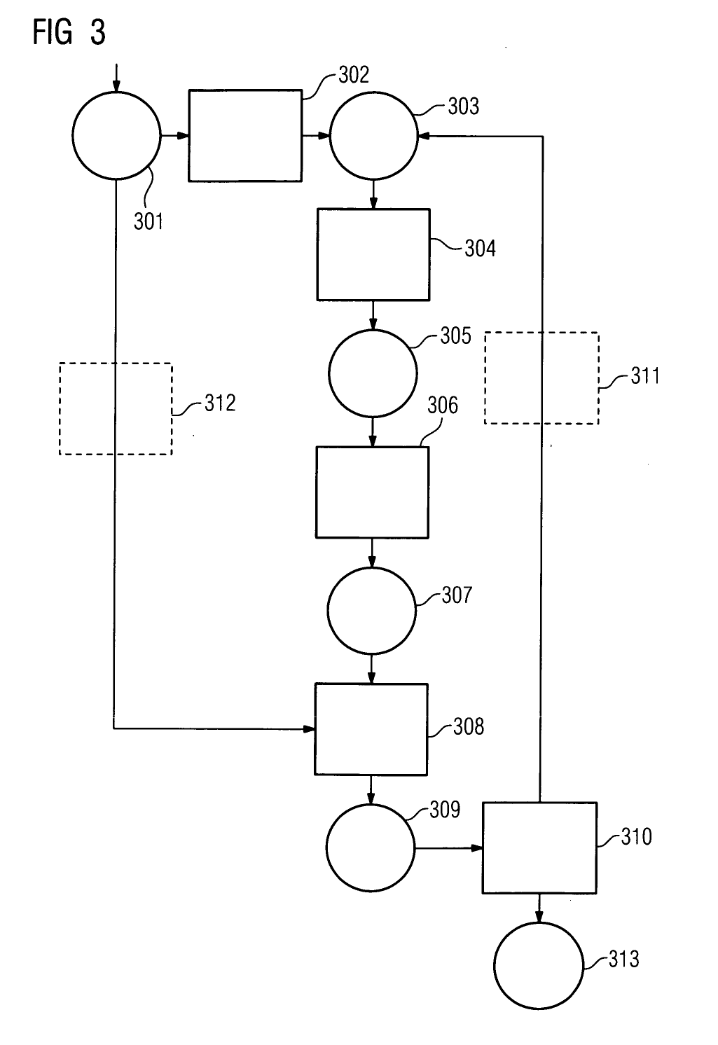

As illustrated in FIG. 3, this transformation can be used to reformulate the above described algorithm as follows:

The measured projections 301 are copied into a memory, which contains the corrected projections 303, in step 302. Even when they are not actually corrected at the beginning of the iteration and correspond to the measured projections 301—the corrected projections 303 are subsequently back projected onto the object in step 304. The image 305 of the object is obtained as a result. The forward projections 307 are calculated from the object thus reconstructed, the image 305, in step 306 following thereupon.

Thereafter, the difference between the calculated and the measured projections is formed in step 308 and output as difference projections 309. A decision is made in step 310 as to whether the difference between the calculated and the measured projections is small enough, or whether a sufficiently large number of iterations have been passed.

If this is not the case, these difference projections 309 are used to correct the corrected projections 303, for which purpose the difference projections 309 are mostly added to the corrected projections. Subsequently, the result, the corrected projections 303, is back projected again onto the image in step 304, the projections of the image are determined etc. This iteration is also repeated until the difference projection is sufficiently small, or a specific number of iterations has been reached. The image 313 is subsequently present in the memory.

A substantial difference from the conventional implementation consists in that the correction is not performed on the image, but on the projections.

An advantage of at least one embodiment of this method is yielded as follows:

Both the forward projections and the back projections can be carried out in a voxel-based or pixel-based fashion—depending on the calculation of volume displays or plane tomograms. Subsequently, the discussion will mention only voxels, these also being pixels in the case of the plane display. Thus, during the back projection, the value for an individual voxel can be determined independently of the other voxels, and the back projection can be serialized with reference to the voxels. The same holds for the projection.

All the projections can be calculated in a voxel-based fashion. All that is required for this purpose is the value of the individual voxel. The projection of the entire object follows from the summation of the individual projections of the various voxels. It is possible in this way to start calculating the projections as soon as the first voxel is calculated, while the other voxels remain to be calculated by the back projection.

While the forward projections of the last voxel are still being calculated, it is possible at the same time to calculate the back projected values of the next voxel. Forward projections and back projections can be carried out in parallel in this way. There is only an offset of one projection of a voxel between the two calculation steps, and this constitutes a negligible time period in view of the size of the calculated objects, currently 512³ voxels.

The reconstructed image 313 can thus either be stored during the back projection of the corrected projections within the iteration and be read out of the memory after truncation of the iteration, or it is determined by way of a further back projection of the corrected projections.

On the basis of this fundamental structure, the difference projection can be ramp filtered in order to accelerate the convergence of at least one embodiment of the iteration method. This optional additional step 311 is illustrated by dashes in FIG. 3. As an alternative, it is also possible to apply an optional ramp filter 312 to the measured projections before the subtraction.

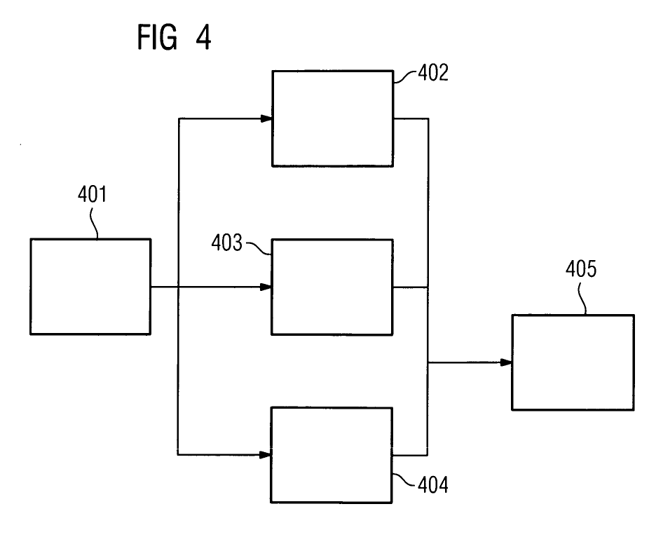

Since the forward projection mostly requires more time than the back projection, the calculation of the forward projection is distributed over a number of arithmetic logic units. As illustrated in FIG. 4, in this process the calculation of the projection of the new pixel is allocated to a free arithmetic logic unit by a distributor unit. In this process, the distributor unit 401 receives the request to have a projection calculated. The distributor unit 401 determines thereupon which of the arithmetic logic units 402 to 404 is currently not being used and passes the request on to one of the free arithmetic logic units that then carries out the calculation and makes the result of the calculation 405 available for further processing. A distribution with 3 arithmetic logic units is illustrated in FIG. 4. However, the number can vary and be adapted to the respective application.

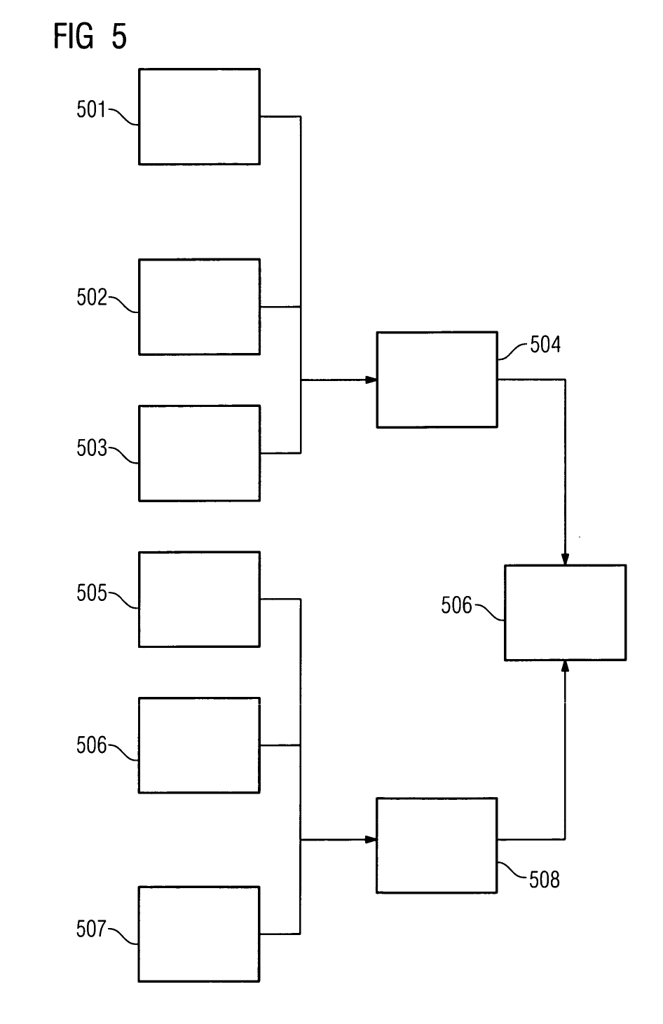

An equally rapid calculation of back projection and forward projection is possible as an alternative by combining the back projection of various corrected projections into one arithmetic logic unit, as is shown in FIG. 5. There, the back projection of 6 projections 501 to 503 and 505 to 507 with the aid of two arithmetic logic units 504 and 508 is illustrated. Each arithmetic logic unit is assigned specific projections that it must process. If the arithmetic logic unit receives the instruction to carry out a back projection, it takes the values of the first projection assigned to it and calculates the back projection. It subsequently processes the second projection etc until all the projections assigned to it are finally processed.

The results of the respective back projection are summed up in an internal memory. Once this is done, the overall result of this arithmetic logic unit is transmitted to an arithmetic logic unit 506 that carries out the summation of the results of all the upstream arithmetic logic units 504 and 508. This function can also be executed in the implementation by one of the upstream arithmetic logic units.

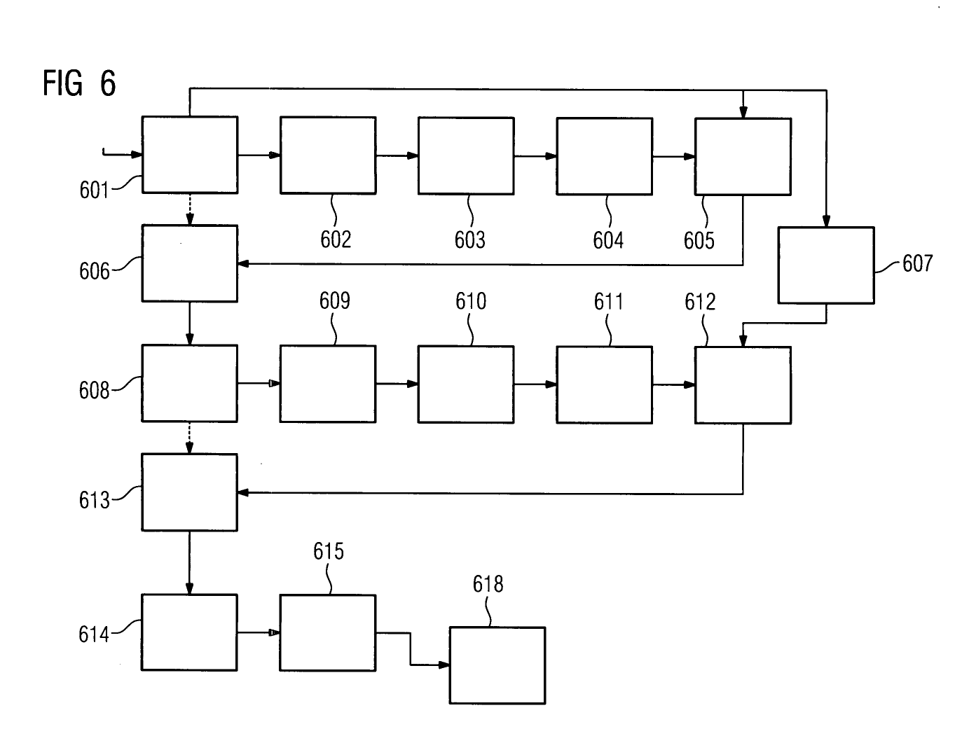

If there is now a limited number of arithmetic logic units available, the calculation of a number of projections is also possible on one arithmetic logic unit. Furthermore, the calculation of the individual iterations can be implemented on various arithmetic logic units. A virtually simultaneous calculation of a number of reconstructions is possible owing to the pipeline structure thereby produced. This is shown by way of example in FIG. 6.

The measured projections 601 are used in a back projection step 602 to determine a first tomographic representation 603 from which projections are subsequently calculated again in a projection step 604. Thereafter, the difference between the calculated and the measured projections is calculated in step 605. The sum 606 of this difference and the measured projections is provided to the second iteration as input data 608. The measured projections 601 are simultaneously copied into a buffer 607.

The back projector 609 now executes the back projection of the projections 608 first corrected. The result is the tomographic representation 610 from which, in turn, projections are calculated by the projector 611. The difference 612 is now formed from these calculated projections and the copied projections 607. This difference is subsequently added in 613 to the firstly corrected data 608 and leaves the sum 614.

The final tomographic representation is calculated from this sum 614 in FIG. 6 in a further back projection step 615.

It would likewise be possible to carry out yet further iterations, the corrected data and the unchanged measured projections being made available as input data for calculating the difference to the respective iteration step. The advantage of this arrangement is that the measured projections are copied into the buffer 607 after the first iteration, the arithmetic logic units that participated in the calculation of the first iteration already being able to begin a new reconstruction while downstream arithmetic logic units are still processing the last reconstruction. The calculation can be accelerated as described above within an iteration presented here.

Since the computational operations are mostly simple calculations, an acceleration is possible without any problem by way of special hardware of any type. It is likewise possible to use a multiprocessor system, a cluster or a network.

It goes without saying that the above-mentioned features of embodiments of the invention can be used not only in the respectively specified combination, but also in other combinations or on their own, without departing from the scope of the invention.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDS; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed:

1. A method for the iterative analytical reconstruction of a tomographic representation of an object from projection data of a moving radiation source through the object onto a detector, the method comprising:
iteratively undertaking corrections with the aid of back projections of the object to be represented from calculated projection data, the corrections being performed on the projections, to thereby reconstruct a tomographic representation of the object.

2. The method as claimed in claim 1, wherein
projections of the object are recorded and at least one representation of the object is back projected,
forward projections are calculated from the at least one tomographic representation of the object,
the recorded projections and the forward projections are compared with one another,
the difference values appearing here between the recorded projections and the calculated forward projections are used as correction values for a corrected projection, and
subsequently, the corrected projections are used for renewed calculation of a tomographic representation of the object, forward projections therefrom and the difference values between the recorded projections and the calculated forward projections and the corrected projection is corrected therewith, until absolute values of the difference values or the number of the iterations reaches a respectively prescribed maximum value.

3. The method as claimed in claim 1, wherein the correction is performed exclusively on the projections.

4. The method as claimed in claim 1, wherein the back projections and the forward projections are carried out in parallel and in a fashion offset by channel.

5. The method as claimed in claim 1, wherein the back projections and the forward projections are carried out in parallel and in at least one of a voxel-wise and pixel-wise fashion.

6. The method as claimed in claim 1, wherein difference projections are calculated in the comparison between the recorded projections and the calculated forward projections, and the difference projections are ramp filtered before the correction of the corrected projections.

7. The method as claimed in claim 1, wherein when calculating the back projections of various corrected projections use is made of a number of arithmetic logic units that is smaller than the number of corrected projections performed.

8. The method as claimed in claim 1, wherein when calculating the back projections of various corrected projections use is made of a number of arithmetic logic units that is the same as the number of corrected projections performed.

9. The method as claimed in claim 1, wherein the calculation of the back projections of consecutive voxels or pixels is carried out on various arithmetic logic units.

10. The method as claimed in claim 1, wherein the calculation of the projections is performed by a number of arithmetic logic units that is smaller than the number of projections to be calculated.

11. The method as claimed in claim 1, wherein the calculation of the projections is carried out by a number of arithmetic logic units that is the same as the projections to be calculated.

12. The method as claimed in claim 1, wherein the calculation of the projections of consecutive voxels is carried out on various arithmetic logic units.

13. The method as claimed in claim 1, wherein projections are obtained from ultrasound imaging.

14. The method as claimed in claim 1, wherein projections are obtained from magnetic resonance imaging.

15. The method as claimed in claim 1, wherein projections are obtained from x-ray imaging.

16. The method as claimed in claim 1, wherein projections are obtained from optical imaging.

17. A computer readable medium including program segments for, when executed on a computer device of a tomography unit, causing the tomography unit to implement the method of claim 1.

18. A tomography unit for iterative analytical reconstruction of a tomographic representation of an object from projection data of a moving radiation source through the object onto a detector, the tomography unit comprising:
the radiation source;
the detector; and
means for iteratively undertaking corrections with the aid of back projections of the object to be represented from calculated projection data, the corrections being performed on the projections, to thereby reconstruct a tomographic representation of the object.

19. The tomography unit as claimed in claim 18, wherein projections are obtained from ultrasound imaging.

20. The tomography unit as claimed in claim 18, wherein projections are obtained from magnetic resonance imaging.

21. The tomography unit as claimed in claim 18, wherein projections are obtained from x-ray imaging.

22. The tomography unit as claimed in claim 18, wherein projections are obtained from optical imaging.
